(19) United States
(12) Patent Application Publication
(10) Pub. No.: US 2008/0015643 A1
(43) Pub. Date: Jan. 17, 2008

(54) IMPLANTABLE ELECTRODE DEVICE

(76) Inventors: Klaus Bartels, Berlin (DE); Wolfgang Geistert, Rheinfelden (DE)

Correspondence Address: DEWITT ROSS & STEVENS S.C., 8000 EXCELSIOR DR, SUITE 401, MADISON, WI, 53717-1914, US

(21) Appl. No.: 11/753,281

(22) Filed: May 24, 2007

(30) Foreign Application Priority Data
Jul. 12, 2006 (DE) 10 2006 032 240.1

(51) Int. Cl. A61N 1/05

(52) U.S. Cl. 607/3

(57) ABSTRACT

An implantable electrode device, in particular an ICD electrode device, comprises an electrode main body and at least one electrode situated thereon, the electrode including a wire material forming undercuts and/or intermediate spaces between its wires, such as a wire braid or an open wire coil. A medication depot filling is introduced into the under-cuts and/or intermediate spaces.

### FIELD OF THE INVENTION

The present invention relates to implantable electrode. In particular, the present invention relates to so-called ICD electrode probes for implantable defibrillators and cardioverters, which have at least one shock electrode.

### BACKGROUND OF THE INVENTION

ICD electrodes are typically implemented as wire braids or open wire coils, the wire being able to have various cross-sections, such as round, flat, crowned, convex, concave, and the like. This wire material forms undercuts and intermediate spaces between its wires and is generally applied to a carrier referred to as the electrode main body.

The implantable electrode device itself may have various constructions. Thus, the following are known from the prior art:
(1) electrode devices which are insertable transvenously and placeable in a ventricle or in the coronary sinus, having an essentially round cross-section and one or more cylindrical shock electrodes;
(2) intracostally placeable electrode devices having an essentially round cross-section and one or more cylindrical shock electrodes, which may be situated on multiple finger-like projections; or
(3) subcutaneously or epicardially placeable electrode devices having a planar implementation of the shock electrodes.

Such electrode devices are described, for example, in the publications U.S. Pat. No. 5,324,328 A, U.S. Pat. No. 5,571,163 A, and WO 02/22208 A2.

On the background of the present invention, it is to be noted that the contact between wire coil and/or wire braid of the shock electrodes on one hand and bodily tissue on the other hand may result in irritations, which may in turn cause immunological reactions, such as inflammations or connective tissue growth. The latter results in ingrowth of the electrode with the consequence that it may only be removed again with difficulty if necessary.

If a coiled shock electrode is located in the blood stream, in contrast, blood clotting and thrombosis may occur. The situation of a shock delivery via the electrode is especially problematic in this case, because clots may be caused on the undercuts, i.e., on the sides of the wires of the shock electrode facing away from the tissue.

An array of measures are known from the prior art for preventing or reducing the reactions explained above. Thus, reducing the ingrowth of the electrode device by coatings using a PTFE material, for example, is described in the publications U.S. Pat. No. 5,931,862 A, U.S. Pat. No. 6,546,292 B1, and US 2003/0023294 A1. Coatings of this type have pores which are too small to allow cells to grow in, but are permeable to liquids. Thus, ions may pass the coating and a current flow is made possible. However, it is disadvantageous that in this way the active surface of the electrode is significantly reduced and the defibrillation threshold thus increases. Furthermore, it is problematic that such coatings may detach and reach the bloodstream as foreign bodies.

The above-mentioned negative reactions may also be avoided or reduced by delivering medications. In this context, U.S. Pat. No. 4,506,680 A1 discloses an electrode device having a medication depot at the electrode tip. The medication is housed in a polymer stopper, which is seated in a cavity in the electrode tip. When bodily fluid is applied to the stopper, it swells up and releases the medicinal active ingredient in the stopper. The active ingredient then reaches the body via a porous coating matrix on the electrode tip. This medication depot is disadvantageous because it represents a component to be mounted separately, and it is located relatively far from the actual shock electrode on the tip.

It is known from above-mentioned U.S. Pat. No. 5,571,163 A and U.S. Pat. No. 5,324,324 A that anti-inflammatory medications may be administered by a coating of the electrode tip and/or the defibrillation electrode. The medications are embedded in a polymer. The disadvantage of these coatings is that they contain a relatively small quantity of medicinal active ingredient and, in addition, they only deliver this medication over a relatively short period of time, because they dissolve and/or detach relatively rapidly. In addition, the coating of the defibrillation electrode also significantly prevents the current flow here, because of which the defibrillation threshold increases.

### SUMMARY OF THE INVENTION

Proceeding from the problems described, the present invention is based on the object of improving an electrode device in such a way that a uniform medication delivery, which occurs located as close as possible to the electrode, is achievable over the longest possible period of time.

This object is achieved by the medication depot filling in the undercuts and/or intermediate spaces of the wire material. The medication depot is quasi-integrated in the electrode by this embodiment, in that small gaps are left between the individual wires of the wire braid and/or the individual turns of the wire coil, which are filled with a flexible plastic carrying the medication to be administered. The medication depot filling is thus simultaneously used for stabilization and fixing of the wire braid and/or the wire coil while simultaneously maintaining the flexibility of the electrode. The problematic undercut areas of the electrode are also filled up by the medication depot filling and are thus no longer harmful.

The medication depot filling is more preferably produced on the basis of a flexible plastic material as a carrier, which contains a medicinal active ingredient finely divided therein and releasable therefrom.

An especially body-compatible and effective embodiment of the electrode device is provided if the medication depot filling completely fills up the space between wire material and/or the wire material has its rear side embedded in the medication depot filling. On one hand, the electrically active surface of the wire material remains practically completely maintained, which keeps the defibrillation threshold low. On the other hand, wire material is optimally enclosed by material which delivers active ingredient, so that a well-dosed medication delivery in immediate proximity to the traumatically problematic shock electrode is achieved.

The plastic material of the medication depot filling is preferably a biocompatible polymer matrix material, such as silicone, polyurethane, or a composite made of these two materials.

The medicinal active ingredient may, for example, be a steroid, such as dexamethasone acetate, dexamethasone sodium phosphate, or beclomethasone and thus an antiinflammatory active ingredient, a heparin and thus an anticlotting active ingredient, sirolimus, paclitaxel, or a magnesium alloy and thus an antiproliferative medication, or a combination of the preceding active ingredients.

In principle, the carrier polymer for the medication depot filling is formulated in such a way that it is sufficiently permeable so that the mixed in medicinal active ingredient may be dissolved out over time. In particular if small quantities of medication are mixed in, this process may be supported if the medication depot filling contains a biodegradable component, such as a salt, a sugar, a polylactate, or a gel. If this component dissolves in the body, microscopic channels are formed in the medication depot filling, through which the active ingredient components lying deeper in the polymer layer may better penetrate outward and be delivered.

Further features, advantages, and details of the present invention result from the following description of exemplary embodiments on the basis of the attached drawing.

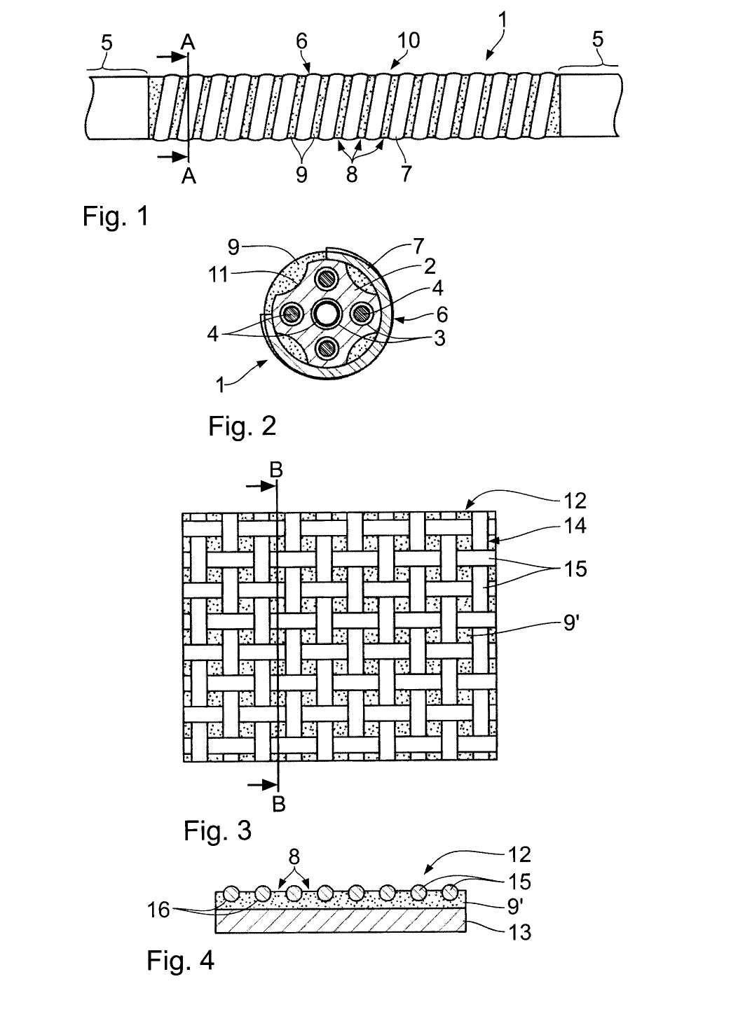

FIG. 1 shows a detail side view of an ICD electrode device,

FIG. 2 shows a cross-section of the electrode device along section line A-A in FIG. 1,

FIG. 3 shows a detail top view of a planar defibrillation electrode, and

FIG. 4 shows a section along section line B-B in FIG. 3 of the defibrillation electrode.

FIGS. 1 and 2 show an ICD electrode device, which is implantable in the heart, having an elongate, tubular electrode body 1. This has a carrier tube 2 as a core, in which multiple lumens 3 run for the passage of the electric supply lines 4 for the shock and stimulation electrodes of the ICD electrode device, which are not shown in greater detail in FIGS. 1 and 2.

An open wire coil 6 is wound between two outer tube sections 5 on the carrier tube 2, which comprises externally concavely bulging flat wire material 7. Helical intermediate spaces 8 are left open between the individual turns of the wire coil 6, which are completely filled up by a medication depot filling 9. Therefore, there are no undercuts on the surface of the electrode body in the area of the shock electrode 10, though the crowned exteriors of the flat wire material 7 may protrude somewhat.

As shown in FIG. 2, the carrier tube 2 has longitudinally axially running hollow grooves 11 in the area of the shock electrode 10, into which the medication depot filling 9 extends.

The latter comprises—as already noted above—a biocompatible polymer matrix material, such as silicone, in which a medicinal active ingredient, such as heparin, is embedded. The active ingredient may escape from the plastic material when impinged by bodily fluid and—in the case of heparin—provide an anticoagulant effect.

The medication depot filling 9 may be formed, for example, by mixing the medicinal active ingredient into the liquid silicone carrier material and then introducing this filling material into the intermediate spaces 8 and the undercuts formed by the hollow grooves 11 and then curing the filling material.

As an alternative to this, the medication depot filling 9 may be produced by introducing the liquid carrier material into the intermediate spaces 8 and hollow grooves 11, curing this material, and introducing the medicinal active ingredient into openings of the cured filling material. The latter may be formed already during the curing by appropriate shaping, by mechanical processing, or by dissolving out a soluble foreign material distributed in the filling material. The medicinal active ingredient may be introduced by immersing the shock electrode 10 having the cured filling material into a medication solution or suspension, or by inserting particles or threads containing active ingredient into the openings produced.

The embodiment shown in FIGS. 3 and 4 represents a planar defibrillation electrode 12, in which a wire braid 14 is fixed over the medication depot filling 9′ on a flexible, leaf-shaped carrier 13. The wire braid 14 is made of woven around wire material 15. The intermediate spaces 8 between the crossing wires and the undercut areas 16 lying under the wires are closed by the medication depot filling 9′. The wire braid 14 is embedded on its rear side in the medication depot filling 9′ in such a way that the latter produces the fixing of the wire braid 14 on the carrier 13.

The statements made in connection with FIGS. 1 and 2 apply for the production and build up of the medication depot filling 9′ of FIGS. 3 and 4, so that further explanations are unnecessary.

What is claimed is

1. An implantable electrode device comprising an electrode main body having:
a. at least one electrode situated on the electrode main body, the electrode including one or more leads with at least one of
(1) undercuts about the leads, and
(2) intermediate spaces between the leads,
b. a medication depot filling in the undercuts and/or intermediate spaces, wherein the medication depot filling contains a releasable medicinal active ingredient.

2. The electrode device of claim 1 wherein the medication depot filling includes a flexible plastic carrier material wherein the medicinal active ingredient is distributed.

3. The electrode device of claim 1 wherein the medication depot filling at least partially surrounds the leads to prevent the definition of any concavities between each lead and the medication depot filling.

4. The electrode device of claim 1 wherein the leads are at least partially embedded within the medication depot filling.

5. The electrode device of claim 1 wherein the leads are defined by a braid.

6. The electrode device of claim 1 wherein the leads are defined by a coil.

7. The electrode device of claim 1 wherein the medication depot filling is a biocompatible polymer matrix material.

8. The electrode device of claim 7 wherein the medication depot filling includes one or more of
a. silicone and
b. polyurethane.

9. The electrode device of claim 1 wherein the medicinal active ingredient in the medication depot filling includes at least one of:
a. an antiinflammatory agent,
b. an anticoagulant agent, and/or
c. an antiproliferative agent.

10. The electrode device of claim 1 wherein the medication depot filling contains a biodegradable component.

11. The electrode device of claim 1 wherein the medication depot filling is formed by the steps of:
a. mixing a medicinal active ingredient into a liquid carrier material;
b. introducing the mixture into the undercuts and/or intermediate spaces; and
c. curing the mixture.

12. The electrode device of claim 1 wherein the medication depot filling is formed by the steps of:
a. introducing a liquid carrier material into the undercuts and/or intermediate spaces;
b. curing the carrier material into an at least semi-solid state; and
c. introducing the medicinal active ingredient into the cured carrier material.

13. The electrode device of claim 12 wherein the cured carrier material has openings defined therein, the openings being formed by at least one of the steps of:
a. shaping the openings into the carrier material during curing;
b. forming the openings into the cured carrier material; and
c. dissolving matter distributed within the carrier material.

14. The electrode device of claim 12 wherein the medicinal active ingredient is introduced into the cured carrier material by at least one of the steps of:
a. immersing the filling material in a medication solution or suspension; and
b. inserting particles or threads containing active ingredient into openings in the cured carrier material.

15. An implantable electrode device having an electrode main body with:
a. a medication depot filling on a surface of the electrode main body, wherein the medication depot filling includes a medicinal active ingredient;
b. an electrical lead at least partially situated on the surface of the electrode main body, wherein:
(1) at least a portion of the electrical lead abuts the medication depot filling, and
(2) less than half of the surface area of the electrical lead is exposed on the surface of the electrode main body.

16. The implantable electrode device of claim 15 wherein the electrical lead is at least substantially embedded within the surface of the electrode main body, wherein less of the electrical lead extends above the surface of the electrode main body than below the electrode main body.

17. The implantable electrode device of claim 15 wherein an angle defined by the adjoining surfaces of the electrical lead and the medication depot filling is greater than 90 degrees at all areas wherein the electrical lead abuts the medication depot filling.

18. An implantable electrode device having an electrode main body with:
a. a surface at least partially defined by a medication depot filling, wherein the medication depot filling includes a medicinal active ingredient;
b. an electrical lead substantially embedded within the surface of the electrode main body, wherein less of the electrical lead extends above the surface of the electrode main body than below the electrode main body.

19. The implantable electrode device of claim 18 wherein less than half of the surface area of the electrical lead is exposed on the surface of the electrode main body.

20. The implantable electrode device of claim 18 wherein an angle defined by any adjoining surfaces of the electrical lead and the medication depot filling is greater than 90 degrees at all areas wherein the electrical lead extends above the surface of the electrode main body.
